(19) United States
(12) Patent Application Publication
(10) Pub. No.: US 2008/0292718 A1
(43) Pub. Date: Nov. 27, 2008

(54) Method for Suppression Appetite and Food Intake

(76) Inventors: Igor Anatolievich Pomytkin, Moscow (RU); Sergey Pavlovich Soloviev, Moscow (RU)

Correspondence Address: NOTARO AND MICHALOS, 100 DUTCH HILL ROAD, SUITE 110, ORANGEBURG, NY, 10962-2100, US

(21) Appl. No.: 12/096,462

(22) PCT Filed: Dec. 12, 2005
(86) PCT No.: PCT/RU2005/000639

§ 371 Date: Aug. 12, 2008

(51) Int. Cl. A61K 33/00; A61P 3/04

(52) U.S. Cl. 424/600

(57) ABSTRACT

The invention relates to method for suppressing appetite and food intake in a mammal in need thereof comprising a step of administering to said mammal an effective amount of water comprising from 99.76 to 99.99 molecular % of isotopologue ¹H₂¹⁶0. Further, invention relates to medical food for suppressing appetite and food intake in a mammal in need thereof which comprises water comprising from about 99.76 to about 99.99 molecular % of isotopologue ¹H₂¹⁶0. Further, invention relates to the use of water comprising from about 99.76 to about 99.99 molecular % of isotopologue ¹H₂¹⁶0 for the manufacture of a medical food for suppressing appetite and food intake in a mammal in need thereof. Because of suppressing appetite and food intake, this invention is particularly useful for treating overweight and obesity in a mammal in need thereof. Preferably, the mammal is human.

### TECHNICAL FIELD

The present invention is in the field of healthcare. More specifically, this invention relates to method for suppression appetite and food intake in mammals, preferably in a human.

### BACKGROUND ART

Nearly two-thirds of adults in the United States are overweight, and 30.5 percent are obese, according to data from the 1999-2000 National Health and Nutrition Examination Survey (NHANES). According to data compiled by the International Obesity Task Force (IOTF), the obesity levels in Europe are from 6.5 in Italy to 20% in Czech Republic. Overweight, and especially obesity, increase risk of many health problems.

Appetite suppressants and peripheral antiobesity agents are major classes of drugs for treating obesity and overweight. These medications have serious side effects and complications. For example, amphetamines have the drawback of being euphoretics with mind altering properties. Thus, there is a great need for a safe, effective appetite suppressant with little or no complications and side effects.

It is known that natural water is a composition of nine water isotopologues (¹H₂¹⁶O, ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O) formed by stable isotopes of hydrogen (¹H and ²H) and oxygen (¹⁶O, ¹⁷O, ¹⁸O), wherein the level of light water isotopologue ¹H₂¹⁶O is about 99.7317 molecular % (Vienna Standard Mean Ocean Water, VSMOW), and wherein total level of all eight heavy isotopologues comprising at least one heavy isotopes ²H, ¹⁷O, or ¹⁸O is about 0.2683% (e.g. 0.199983% ¹H₂¹⁸O, 0.0372% ¹H₂¹⁷O, 0.031069% ¹H²H¹⁶O, 0.0000623% ¹H²H¹⁸O, and 0.0000116% ¹H²H¹⁷O). Rothman et al., J. Quant. Spectrosc. Radiat. Transfer, 1998, 60, 665. Rothman et al., J. Quant. Spectrosc. Radiat. Transfer, 2003, 82, p. 9. The abundance of water isotopologues in natural water slightly varies on Earth district and climatic conditions and is expressed typically as the deviation, δ, relative to the international VSMOW standard. The Earth water maximally enriched by major light water isotopologue ¹H₂¹⁶O was founded in Antarctica (Standard Light Antarctic Precipitation, SLAP), wherein said δ-values of residual heavy isotopes are δ²H −415.5‰, δ¹⁷O −28.1‰, and δ¹⁸O −53.9‰ that corresponds to the 99.757% level of light water isotopologue ¹H₂¹⁶O. R. van Trigt, Laser Spectrometry for Stable Isotope Analysis of Water Biomedical and Paleoclimatological Applications, 2002, Groningen: University Library Groningen, p. 50. Thus, water with the abundance of light water isotopologue H₂¹⁶O more than 99.757 molecular % is not found in nature.

Deuterium depleted water (DDW) is known from the art and is prepared from natural water by industrial procedures providing depletion of heavy isotopologues comprising deuterium, predominantly of ¹H₂¹⁶O (HOD). Since total levels of deuterium-comprising isotopologues in water is below 0.031 molecular %, complete depletion of natural water of deuterium-comprising isotopologues provides water enriched by light water isotopologue ¹H₂¹⁶O to the level never more than 99.76 molecular %. Thus, water with level of light water isotopologue ¹H₂¹⁶O more than 99.76% is unknown from the art and can be prepared in industrial scale by methods providing depletion of natural water of heavy isotopologues comprising isotopes ¹⁷O and ¹⁸O.

We discovered that water with isotopologue ¹H₂¹⁶O level more than 99.76 molecular % is useful for suppression appetite and food intake in mammals in need thereof.

It is an object of the present invention to provide a method for suppressing appetite and food intake in a mammal in need thereof comprising a step of administering to said mammal an effective amount of water comprising from 99.76 to 99.99 molecular % of isotopologue ¹H₂¹⁶O.

It is an object of the present invention to provide a medical food for suppressing appetite and food intake in a mammal in need thereof which comprises water comprising from about 99.76 to about 99.99 molecular % of isotopologue ¹H₂¹⁶O.

It is an object of the present invention to provide the use of water comprising from about 99.76 to about 99.99 molecular % of isotopologue ¹H₂¹⁶O for the manufacture of a medical food for suppressing appetite and food intake in a mammal in need thereof.

### BRIEF DESCRIPTION OF THE DRAWINGS

FIG. 1 is a schematic side view of an apparatus for the manufacturing the water comprising from 99.76 to 99.99 molecular % of isotopologue ¹H₂¹⁶O.

### DISCLOSURE OF INVENTION

The present invention provides a method for suppressing appetite and food intake in a mammal in need thereof comprising a step of administering to said mammal an effective amount of water comprising from 99.76 to 99.99 molecular % of isotopologue ¹H₂¹⁶O.

As used herein, the term “isotopologue” is in accordance with IUPAC Compendium of Chemical Terminology 2nd Edition (1997) and refers to a molecular entity that differs only in isotopic composition (number of isotopic substitutions), e.g. ¹H₂¹⁶O, ¹H²H¹⁶O, ¹H₂¹⁸O.

The water of the invention comprising from 99.76 to 99.99 molecular % of isotopologue ¹H₂¹⁶O can be prepared by a variety of industrial procedures. Such procedures include, but are not limited to, burning molecular hydrogen with molecular oxygen with desired low heavy isotope content, or industrial procedures providing purification of natural water of heavy isotopologues comprising heavy isotopes ²H, ¹⁷O, and ¹⁸O. Preferably, the water of the invention is prepared by highly-effective distillation of natural water.

According to present invention, the water of the invention comprises from 99.76 to 99.99 molecular % of isotopologue ¹H₂¹⁶O and up to 100 molecular % of residual isotopologues. As used herein, the term “residual isotopologues” refers to ¹H₂¹⁷O, ¹H₂¹⁸O, ¹H₂²H¹⁶O, ¹H²H¹⁷O, ¹H²H¹⁸O, ²H₂¹⁶O, ²H₂¹⁷O, and ²H₂¹⁸O. In the invention, relative amounts of particular heavy isotopologues could vary depending upon the procedure of the preparing the water of the invention, but the total sum of residual isotopologues formed by heavy isotopes ²H, ¹⁷O, [symbol] ¹⁸O should not exceed 0.01 to 0.24 molecular %. The amounts of heavy isotopes in the residual isotopologues could vary from 0.01 ppm to 155 ppm for ²H, 1 to 360 ppm for ¹⁷O, and 1 to 2000 ppm for ¹⁸O, but the total sum of the residual isotopologues formed by these amounts of heavy isotopes should not exceed 0.01 to 0.24%.

In practicing the method of the invention, the effective amount of water comprising from 99.76 to 99.99 molecular % of isotopologue ¹H₂¹⁶O can be administered in a variety of routes including oral (e.g. through gastrointestinal tract or oral mucosa), intranasal, topical, rectal, by inhalation spray, or parenteral (e.g. subcutaneous, intravenous, or intramuscular injections). Preferably, the effective amount water comprising from 99.76 to 99.99 molecular % of isotopologue ¹H₂¹⁶O is administered orally.

Preferably, the effective amount of the water comprising from about 99.76 to about 99.99 molecular % of isotopologue ¹H₂¹⁶O is 0.1 to 50 g/kg body weight of a mammal.

As used herein, the term “mammal” refers to any mammal. Nonexclusive examples of such mammals include, but are not limited to, animals such as a dog, a cat, and a horse and a human. Preferably, the mammal is a human.

In practicing the method of the invention, the effective amount of water comprising from 99.76 to 99.99 molecular % of isotopologue ¹H₂¹⁶O isotopologues can be administered in a variety of different dosage forms, i.e., they may be formulated in the form of solutions, spray, liquid aerosols, elixirs, syrups, and the like. Ingredients that can be used for preparing dosage forms of the invention may include, but are not limited to, buffering agents (such as phosphate buffer, carbonate buffer, tris buffer, tartrate buffer, borate buffer, acetate buffer, succinate buffer, or maleate buffer), colorants, flavorants, preservatives, antioxidants, surfactants, and etc.

In practicing the method of the invention, the effective amount of water comprising from 99.76 to 99.99 molecular % of isotopologue ¹H₂¹⁶O can be administered stepwise or simultaneously with other appetite suppressants. Such appetite suppressants include, but are not limited to, benzphetamine, diethylpropion, mazindol, phendimetrazine, and phentermine.

Further, the present invention provides a medical food for suppressing appetite and food intake in a mammal in need thereof which comprises water comprising from about 99.76 to about 99.99 molecular % of isotopologue ¹H₂¹⁶O.

Further, the present invention provides the use of water comprising from about 99.76 to about 99.99 molecular % of isotopologue ¹H₂¹⁶O for the manufacture of a medical food for suppressing appetite and food intake in a mammal in need thereof.

Preferably, the medical food for suppressing appetite and food intake is drinking water or beverage. Preferably, the medical food of the invention is drinking water manufactured by saturation of the water of the invention with carbon dioxide or/and inorganic salts typically abandoned in natural drinking water. The examples of such salts include, but are not limited to, sodium chloride, sodium bicarbonate, calcium chloride, magnesium sulfate, etc.

Because of suppressing appetite and food intake, this invention is particularly useful for treating overweight and obesity in a mammal in need thereof. Nonexclusive examples of the mammal include overweight and obese humans and pets like as cats and dogs.

The following examples are presented to demonstrate the invention. The examples are illustrative only and are not intended to limit the scope of the invention in any way.

### EXAMPLE 1

This example demonstrates the method for producing the water of the invention.

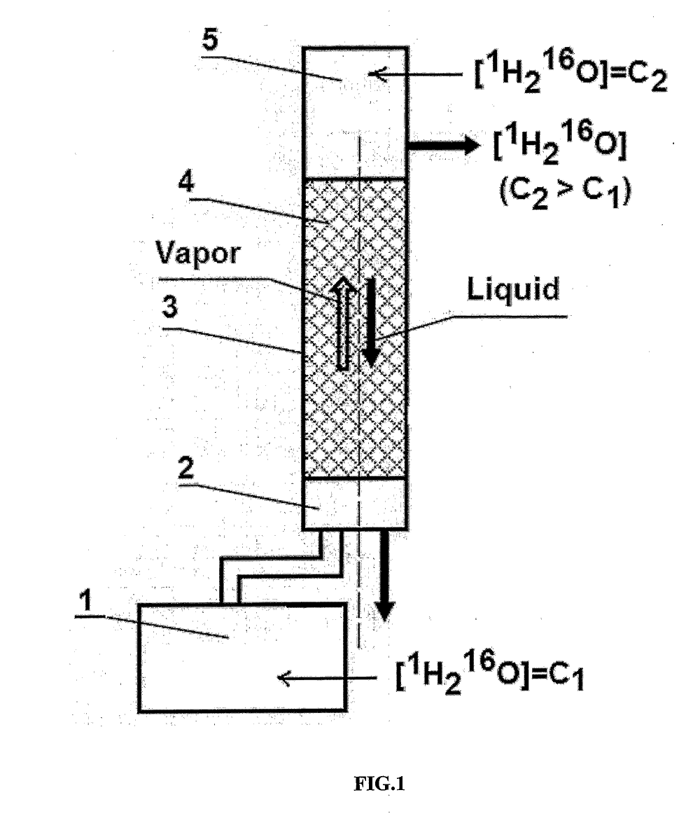

Water comprising from 99.76 to 99.99 molecular % of isotopologue ¹H₂¹⁶O is prepared by distillation of natural water comprising 99.73% of isotopologue ¹H₂¹⁶O with using the apparatus of FIG. 1 under temperature 60° C. and pressure 0.2 bars. The process of the distillation comprises evaporating natural water comprising 99.71% (C₁) of isotopologue ¹H₂¹⁶O in boiling means 1 to produce water vapor; supplying the water vapor to the bottom 2 of distillation column 3; carrying out vapor-liquid contact between a descending liquid and an ascending vapor mainly on the surface of the contact device 4 (e.g. structured or random packing) within the distillation column, at which time the liquid and the vapor flow in mutually opposite directions over the surface of the contact device along a main flow direction which is along a direction of the column axis; condensing water vapor with concentration of isotopologue ¹H₂¹⁶O from 99.76% to about 99.99% (C₂) on condenser 5 installed on upper bound of the distillation column 3; and collecting a part of condensate as condensed water comprising from 99.76% to 99.99% of isotopologue ¹H₂¹⁶O (C₂>C₁). Water comprising from 99.76 to 99.99 molecular % of isotopologue ¹H₂¹⁶O is used in the examples of the invention.

### EXAMPLE 2

This example demonstrates the method for suppressing appetite and food intake.

Male Wistar rats were used. The rats were assigned into two groups: a control rats (n=10) and experimental rats (n=10). Experimental rats received water (99.80 molecular % of isotopologue ¹H₂¹⁶O) as water for drinking for 24 before and during the time of the experiment. Control rats received control water with natural isotopes content (99.73 molecular % of isotopologue ¹H₂¹⁶O). Both waters were received ad libitum. Average water intake in both groups was about 20 ml per rat per day. The effect of waters on feeding motivation was assessed by lowering intake of food granules (45 mg, P. S. Noyes Company Inc., USA) by rats in test described below.

The rats received food granules in special instrumental camera (Lafayette Instruments Inc., USA). Briefly, after night food deprivation a rat was located in the camera for 50 minutes, wherein rat could receive the food granule after pressing on a special lever. When the rat received the food granule, a light in the camera was switched off and lever was disabled for 17 s (latent period). Rats could receive the food granule after a single pressing on the lever at the first day, after two consequent pressings at day 2, after four pressings at day 3, after eight pressings at day 4, after sixteen pressings at day 5, after thirty two at day 6, after sixty four consequent pressings at day 7, and after one hundred twenty eight pressings at day 8. The number of food granules received by control and experimental rats was measured. The data are presented as mean ±SD (n=10) as number of food granules per rat in the day of the experiment in table 1. Table 1 demonstrates that water of the invention is effective for suppressing appetite and food intake (3-fold decrease in food intake by rats at day 7) as compared to control.

### EXAMPLE 3

This example demonstrates medical food for suppressing appetite and food intake.

The medical food as described in Table 2 was manufactured as follows: salts (calcium chloride, magnesium chloride, and sodium bicarbonate) in amounts shown in Table 3 were dissolved in the water (99.99 molecular % of isotopologue ¹H₂¹⁶O) under room temperature and, then, resulted product was bottled in bottles of 330 ml volume.

### EXAMPLE 4

This example demonstrates beverage for suppressing appetite and food intake.

The beverage is manufactured by saturation of the composition of example 3 with carbon dioxide and bottling the final product.

### EXAMPLE 5

This example demonstrates that medical food of example 4 is useful for reducing weight in humans due to suppressing appetite and food intake.

Two overweight male humans with mean BMI 29.0 kg/m² have received 100 ml/day of the beverage of example 4 at 60 min after eating for a period of 30 days. At the end of the experiment the mean BMI was reduced to the 26.8 kg/m² as a result of suppressing appetite and food intake.

TABLE 1
Number of Food Granules Received by Rats.

| | |
|---|---|
| Pressings per | Number of granules per Rat |

| Day | Granule | Control | Experimental |
|---|---|---|---|
| 4 | 8 | 69.3 ± 29.0 | 68.7 ± 31.9 |
| 5 | 16 | 42.5 ± 25.0 | 29.8 ± 20.7 |
| 6 | 32 | 19.8 ± 13.7 | 11.4 ± 6.5 |
| 7 | 64 | 5.7 ± 5.0 | 1.9 ± 1.9* |
| 8 | 128 | 1.2 ± 2.9 | 0.1 ± 0.3 |

*Denotes statistically significant difference of the control (p < 0.05).

TABLE 2
Medical food for suppressing appetite and food intake.

| | Content, |
|---|---|
| | weight % |
| Water (99.80 molecular % of isotopologue ¹H₂¹⁶O) | 99.953 |
| Calcium chloride | 0.015 |
| Magnesium chloride | 0.007 |
| Sodium bicarbonate | 0.025 |

We claim:

1. A method for suppressing appetite and food intake in a mammal in need thereof comprising a step of administering to said mammal an effective amount of water comprising from 99.76 to 99.99 molecular % of isotopologue H₂¹⁶O.

2. The method according to claim 1, wherein the effective amount water comprising from 99.76 to 99.99 molecular % of isotopologue ¹H₂¹⁶O is administered orally.

3. The method according to claim 1, wherein the effective amount of the water comprising from about 99.76 to about 99.99 molecular % of isotopologue ¹H₂¹⁶O is 0.1 to 50 g/kg body weight of a mammal.

4. The method according to claim 1, wherein the effective amount of the water comprising from about 99.76 to about 99.99 molecular % of isotopologue ¹H₂¹⁶O is administered for a period of 1 day or longer.

5. The method according to claim 1, wherein the mammal is a human.

6. A medical food for suppressing appetite and food intake in a mammal in need thereof which comprises water comprising from about 99.76 to about 99.99 molecular % of isotopologue ¹H₂¹⁶O.

7. The medical food according to claim 6, wherein the mammal is a human.

8. The use of water comprising from about 99.76 to about 99.99 molecular % of isotopologue ¹H₂¹⁶O for the manufacture of a medical food for suppressing appetite and food intake in a mammal in need thereof.

9. The use according to claim 8, wherein the mammal is a human.
